(19) United States
(12) Patent Application Publication
(10) Pub. No.: US 2026/0306425 A1
(43) Pub. Date: Oct. 8, 2026

(54) MATERIALS AND METHODS FOR TREATING FUNGAL INFECTIONS

(71) Applicant: Virginia Tech Intellectual Properties, Inc., Blacksburg, VA (US)

(72) Inventors: Mohamed N. SELEEM, Blacksburg, VA (US); Ehab SALAMA, Blacksburg, VA (US)

(21) Appl. No.: 19/640,871

(22) Filed: Apr. 7, 2026

Related U.S. Application Data
(60) Provisional application No. 63/785,411, filed on Apr. 8, 2025.

(51) Int. Cl. A61K 31/122; A61K 31/7048; A61P 31/10

(52) U.S. Cl. CPC A61K 31/122; A61K 31/7048; A61P 31/10

(57) ABSTRACT

The subject invention provides compositions and methods for treating fungal infections using combination therapy comprising amphotericin B and idebenone. Idebenone has been identified as a potent potentiator of amphotericin B, capable of restoring and enhancing its fungicidal activity against amphotericin B-resistant fungal pathogens, thereby demonstrating a strong synergistic effect. The disclosed compositions and methods offer an effective approach for improving antifungal therapy, particularly against drug-resistant pathogens, by enhancing the efficacy of amphotericin B while reducing required dosage and associated toxicity.

### CROSS-REFERENCE TO A RELATED APPLICATION

This application claims priority to U.S. Provisional Patent Application No. 63/785,411, filed Apr. 8, 2025, which is incorporated herein by reference in its entirety.

### BACKGROUND OF THE INVENTION

Every year, hundreds of thousands of individuals around the world suffer from consequences associated with invasive fungal infections. Most of these infections are caused by pathogenic species of Candida, Cryptococcus, and Aspergillus. Globally, the challenge of invasive fungal infections is magnified by the scarcity of antifungal drugs and the escalating resistance to existing agents.

First identified in Japan in 2009, the emerging Candida species (Candida auris) (also known as Candidozyma auris) rapidly spread to several countries, leading to considerable morbidity and mortality. Moreover, C. auris presents an additional challenge because of its remarkable capacity not only to survive on surfaces but also to be transmitted across hospital environments through healthcare workers and on medical tools. C. auris is a prime example of how the susceptibility profile of Candida species to conventional antifungal drugs has dramatically shifted. C. auris isolates demonstrate an exceptional resistance to azole antifungals, which has limited the use of this important class of antifungals (90% of isolates are resistant to fluconazole). Unlike other Candida species, C. auris demonstrates a high level of resistance to the potent polyene antifungal, amphotericin B. Echinocandins are the only class of antifungals that are still effective against C. auris but careful monitoring of resistance to this class is necessary. Alarmingly, recent reports have identified pan-resistant Candida auris isolates, demonstrating resistance to all antifungal drugs, in different states across the United States.

Amphotericin B (AmB) stands out as the most comprehensive systemic antifungal since its introduction in the 1950s and remains as a gold standard agent for the treatment of life-threatening invasive fungal infections. While AmB is an extremely effective antifungal and antiprotozoal agent, its clinical application is hampered by significant drawbacks and serious dose-limiting side effects. Notably, dose-dependent renal toxicity restricts its use in many patients. Adding to the complexity, 30% of the emerging fungal isolates, C. auris, display resistance to AmB.

Due to the pronounced adverse reactions associated with AmB, the recommended plasma concentration in clinical settings is restricted to 1-2 μg/mL. Consequently, its effectiveness is compromised when dealing with Candida strains having minimum inhibitory concentration (MIC) values exceeding 1 μg/mL. Additionally, a significant correlation has been identified between therapeutic failure and mortality in cases where AmB was employed for treating infections caused by Aspergillus isolates with MICs of >2 μg/mL. Notably, the majority of patients harboring isolates within this MIC range died.

To mitigate the toxicity associated with AmB, various formulations, including liposomal, colloidal, and lipid complex forms, have been developed. However, these formulations have not completely alleviated the associated side effects. Therefore, new solutions are urgently required to confront the diminished antifungal arsenal, particularly when challenged with highly resistant fungal pathogens such as C. auris.

### BRIEF SUMMARY OF THE INVENTION

The present invention relates to combination antifungal therapies that advantageously enhance the efficacy of existing antifungal agents while reducing associated toxicity. In particular, the invention provides a therapeutic strategy in which a potentiator compound is administered in combination with amphotericin B (AmB) to restore or enhance its antifungal activity against multidrug-resistant fungal pathogens. By improving the effectiveness of AmB, the combination enables the use of lower dosages, thereby minimizing adverse side effects while maintaining or improving therapeutic outcomes.

In a specific embodiment, idebenone (FIG. 1), a synthetic analog of coenzyme Q10 with well-established antioxidant properties, has been found to be a potent enhancer of AmB activity against highly resistant isolates of C. auris.

Advantageously, the combination of AmB and idebenone (IDB) exhibits strong fungicidal activity not only against Candida auris but also against other clinically relevant fungal pathogens, including other species of Candida, Cryptococcus, and Aspergillus. In vivo studies using an infected Caenorhabditis elegans model confirm the enhanced therapeutic efficacy of the combination.

The subject invention provides compositions and methods for treating fungal infections, particularly those caused by drug-resistant organisms. Advantageously, the compositions and methods of the subject invention leverage the synergistic interaction between AmB and IDB.

In certain embodiments, the subject invention provides a pharmaceutical composition comprising an antifungal agent and a potentiator compound that enhances the efficacy of the antifungal agent. In a specific embodiment, the antifungal agent is AmB and the potentiator compound is IDB or an analog thereof.

In certain embodiments, the subject invention provides a composition comprising AmB, IDB or an analog thereof, and optionally, a pharmaceutically acceptable carrier. In a specific embodiment, the composition consists of, or consists essentially of, AmB, IDB or an analog thereof, and optionally, a pharmaceutically acceptable carrier.

In certain embodiments, the subject invention provides a method of treating an infection caused by a fungal pathogen in a subject, the method comprising administering to the subject an effective amount of AmB and IDB, wherein IDB sensitizes the fungal pathogen to AmB.

In some embodiments, the fungal pathogen is selected from Candida spp., Malassezia spp., Cryptococcus spp., Aspergillus spp., Nocardia spp., Saccharomyces spp., Microsporum spp, and Trichophyton spp.

In specific embodiments, the fungal pathogen is Candida spp. selected from Candida albicans, Candida auris, Candida glabrata, Candida parapsilosis, Candida dubliensis, Candida tropicalis, Candida lusitaniaem, Candida pseudoguillerimondi, and Candida krusei. In a specific embodiment, the fungal pathogen is Candida albicans or Candida auris.

In specific embodiments, the fungal pathogen is Cryptococcus spp, such as Cryptococcus neoformans, or Aspergillus spp, such as Aspergillus fumigatus.

In certain embodiments, the subject invention provides a method for restoring or enhancing the fungicidal activity of AmB against fungal pathogens in a subject, the method comprising co-administering IDB to the subject in need thereof with AmB.

In a specific embodiment, the fungal pathogen is Candida albicans, Candida auris, Cryptococcus neoformans, or Aspergillus fumigatus.

In a specific embodiment, the fungal pathogen is a multidrug-resistant fungal pathogen that is resistant to AmB.

In a specific embodiment, the subject is an immunocompromised individual.

### BRIEF DESCRIPTION OF THE DRAWINGS

FIG. 1 shows the Structure of Idebenone and Co-enzyme Q10.

FIG. 2A-2B show (A) Time-kill analysis of amphotericin B (AmB), idebenone (IDB) or a combination of both drugs against C. auris AR0390. (B) Spot assay of treated cultures of C. auris AR0390 onto YPD agar plates.

FIG. 3 shows in vitro cytotoxicity of AmB in the presence or absence of IDB against monkey kidney (Vero) cells

FIG. 4 shows in vivo efficacy of AmB/IDB combination against multidrug-resistant C. auris in a C. elegans infection model.

FIG. 5 shows in vivo optimization of AmB dose in murine model of C. auris infection. Groups of female CD-1 mice (5 mice per group) were infected with AmB-resistant C. auris AR0390 (3.3×10⁷ CFU/mouse) and treated with vehicle control (untreated), or different dose of AMB (1,0.75, 0.5 and 0.25 mg/kg), daily for two days. On day three the kidney C. auris burden (log CFU) was determined and each mouse was represented by a dot in the graph. The data were analyzed via a one-way analysis of variance (ANOVA) using post-hoc Dunnett's test for multiple comparisons. The asterisk (*) indicates a statistically significant difference (P<0.05) compared to the untreated control.

FIGS. 6A-6B show A) In vivo optimization of AmB dose in C57BL/6 mouse model of Cryptococcal infection. B) In vivo optimization of A. fumigatus 13073 inoculum in CD-1 mouse model.

### DETAILED DESCRIPTION OF THE INVENTION

The subject invention provides compositions and methods for treating fungal infections, specifically, infections caused by multidrug-resistant fungal pathogens. The subject invention provides a therapeutic strategy by using a potentiator compound to resensitize multidrug-resistant fungal pathogens to existing antifungal agents at lower dosages to minimize adverse side effects while maintaining or improving therapeutic outcomes.

The present invention provides antifungal compositions and methods that utilize idebenone (IDB) as a potentiating agent to restore or enhance the activity of amphotericin B (AmB) against fungal pathogens, in particular, multidrug-resistant fungal pathogens, including Candida auris. The subject invention arises from the discovery that IDB is capable of re-sensitizing resistant fungal strains to AmB, thereby improving antifungal efficacy while enabling reduced dosing of AmB and mitigating its associated toxicity.

Idebenone is a synthetic analog of coenzyme Q10 characterized by improved aqueous solubility and bioavailability resulting from its shorter lipophilic side chain. In addition to its favorable pharmacokinetic properties, IDB exhibits strong antioxidant activity and the ability to support mitochondrial function. The present invention identifies that these properties contribute to its cytoprotective effects in eukaryotic cells, and make it suitable for use in combination therapies that require both efficacy against pathogens and reduced host toxicity.

The disclosed compositions and methods provide a safe and effective therapeutic approach for treating fungal infections, particularly those caused by highly drug-resistant organisms, by combining AmB with IDB to achieve synergistic antifungal activity and improved therapeutic outcomes.

In certain embodiments, the invention provides a cost-effective therapeutic approach by combining AmB with IDB, a relatively low-cost compound compared to other antifungal agents or adjunct therapies. This embodiment is particularly advantageous for use in resource-limited settings, including regions with high fungal disease burden and limited access to expensive treatments.

The subject invention provides innovative antifungal strategies to combat drug-resistant fungal infections and to improve clinical outcomes in the management of fungal diseases.

In certain embodiments, the subject invention provides a pharmaceutical composition comprising an antifungal agent and a potentiator compound that enhances the efficacy of the antifungal agent. In a specific embodiment, the antifungal agent is AmB and the potentiator compound is IDB or an analog thereof.

In certain embodiments, the subject invention provides a pharmaceutical composition comprising AmB, IDB or an analog thereof, and optionally, a pharmaceutically acceptable carrier. In some embodiments, the compositions are formulated to enable reduced dosing of AmB while maintaining efficacy. The term “analog” refers to a molecule that is not identical to IDB, but has analogous functional and structural features.

“Pharmaceutically acceptable carrier” refers to a diluent, adjuvant or excipient with which the one or more active agents disclosed herein can be formulated. Typically, a “pharmaceutically acceptable carrier” is a substance that is non-toxic, biologically tolerable, and otherwise biologically suitable for administration to a subject, such as an inert substance, added to a pharmacological composition or otherwise used as a diluent, adjuvant or excipient to facilitate administration of the composition disclosed herein and that is compatible therewith. Preferably, said carrier and/or diluent is/are selected from water, physiologically acceptable aqueous solutions containing salts and/or buffers and any other solution acceptable for administration to an animal, for example, distilled water, de-ionized water, pure or ultrapure water, saline, phosphate-buffered saline (PBS), solutions containing usual buffers that are compatible with the other components of the composition.

Examples of carriers suitable for use in the pharmaceutical compositions are known in the art and such embodiments are within the purview of the invention. The pharmaceutically acceptable carriers and excipients, including, but not limited to, aqueous vehicles, water-miscible vehicles, non-aqueous vehicles, stabilizers, solubility enhancers, isotonic agents, buffering agents, suspending and dispersing agents, wetting or emulsifying agents, complexing agents, sequestering or chelating agents, cryoprotectants, lyoprotectants, thickening agents, pH adjusting agents, and inert gases. Other suitable excipients or carriers include, but are not limited to, dextran, glucose, maltose, sorbitol, xylitol, fructose, sucrose, and trehalose.

In certain embodiments, the chelating agent may be any pharmaceutically acceptable chelating agent. Suitable chelating agents include, but are not limited to, an aminopolycarboxylic acid such as glycinate, iminodiacetic acid, nitrilotriacetic acid, nitrilotripropionic acid, ethylenediamine tetraacetic acid (EDTA), diethylenetriamine pentaacetic acid, ethylene glycol-bis(P-aminoethyl ether)-/V,/V,/V′,/V′-tetraacetic acid, 1,2-bis(o-aminophenoxy) ethane-/V,/V,/V/,/V/-tetraacetic acid, 1,4,7-triazacyclononane-1,4,7-triacetic acid, 2,2′,2″,2′″-(1,4,7,10-tetraazacyclododecane-1,4,7,10-tetrayl)tetraacetic acid, nicotimamine, ethylenediamine-/V,/V′-bis(2-hydroxyphenylacetic acid), ethylenediamine disuccinic acid, 2-hydroxyethyl-ethylenediamine-triacetic acid, 1,6-diamino-hexamethylene-tetraacetic acid, 1,2-diamino-cyclohexane tetraacetic acid, 0,09-bis(2-aminoethyl)-ethyleneglycol-tetraacetic acid, 1,3-diaminopropane-tetraacetic acid,/V,/V-bis(2-hydroxybenzyl)ethylenediamine-/V,/V-diacetic acid, ethylenediamine-/V,/V9-diacetic acid, ethylenediamine-/V,/V9-dipropionic acid, triethylenetetraamine hexaacetic acid, 7,19,30-trioxa-1,4, 10, 13, 16,22,27,33-octaazabicyclo [l1,l1,l1]pentatriacontane (O-bis-tren), ethylenediamine-/V,/V9-bis(methylenephosphonic acid), iminodiacetic acid,/V,/V-bis(2-hydroxyethyl)glycine, 1,3-diamino-2-hydroxypropane-tetraacetic acid, 1,2-diaminopropane-tetraacetic acid, ethylenediamine-tetrakis(methylenephosphonic acid), N-(2-hydroxyethyl)iminodiacetic acid or triethylenetetramine-hexaacetic acid; deferoxamine, dimercaprol; citrate (e.g. zinc citrate); penicillamine; a bisphosphonate such as etidronate; a macrocyclic polyether; polyaspartic acid; methylglycinediacetic acid; glutamic diacetic acid; gluconic acid; or a pharmaceutically acceptable salt or combination of any of the foregoing.

In certain embodiments, the buffering agent may be any pharmaceutically acceptable buffering agent. Suitable buffering agents include, but are not limited to, tris(hydroxymethyl)aminomethane (Tris), triethylamine, triethylenetetramine, tetraethylethylenediamine, tetramethylenediamine,/V,/V′-diethyl-/V,/V′-bis(sulfopropyl)-ethylenediamine,/V,/V′-diethylpiperazine, piperazine-/V,/V′-bis(alkylsulfonic acids), (/V-morpholino)alkylsulfonic acids (e.g. 2-morpholineethanesulfonic acid or 4-morpholinepropane sulfonic acid), 2-aminoethanol, 2-amino-2-methyl-1-propanol, triethanolamine, 2-amino-2-methyl-1,3-propanediol, bis-(2-hydroxyethyl)imino-tris(hydroxymethyl) methane, 2-dimethylamino-2-methyl-1-propanol, 2-amino-2-ethyl-1,3-propanediol, 1,3-bis(tris [hydroxymethyl]methylamino) propane,/V,/V-bis(2-hydroxyethyl)-2-aminoethanesulfonic acid,/V-[tris(hydroxymethyl)methyl]-3-aminopropanesulfonic acid, /V,/V-bis(2-hydroxyethyl)glycine, /V,/V-bis(2-hydroxyethyl)taurine, diethanolamine,/V-tris(hydroxymethyl)methylglycine, 2,2 bis(hydroxymethyl)-2,29,20-nitrilotriethanol, 1,3 bis(tris [hydroxymethyl]methylamino) propane, acetic acid, adipic acid, ammonia solution, ammonium phosphate, ammonium sulfate, arginine, asparagine, boric acid, calcium carbonate, calcium lactate, calcium phosphate, tribasic, citric acid monohydrate, dibasic potassium phosphate, dibasic sodium phosphate, glycine, histidine, hydroxyethylpiperazine ethane sulfonic acid, lysine acetate, lysine hydrochloride, maleic acid, malic acid, meglumine, methionine, monobasic sodium phosphate, monoethanolamine, monosodium glutamate, phosphoric acid, potassium citrate, potassium metaphosphate, sodium acetate, sodium bicarbonate, sodium bisulfate, sodium borate, sodium carbonate, sodium chloride, sodium citrate dihydrate, sodium lactate, tromethamine or a pharmaceutically acceptable salt or combination of any of the foregoing. In particular embodiments, the buffering agent is tris(hydroxymethyl)aminomethane (Tris), triethylamine, triethylenetetramine, tetraethylethylenediamine, tetramethylenediamine,/V,/V′-diethyl-/V,/V′-bis(sulfopropyl)-ethylenediamine,/V,/V′-diethylpiperazine, piperazine-/V,/V′-bis(alkylsulfonic acids), (/V-morpholino)alkylsulfonic acids, 2-aminoethanol, 2-amino-2-methyl-1-propanol, triethanolamine, 2-amino-2-methyl-1,3-propanediol, bis-(2-hydroxyethyl)imino-tris(hydroxymethyl) methane, 2-dimethylamino-2-methyl-1-propanol, 2-amino-2-ethyl-1,3-propanediol, 1,3-bis(tris [hydroxymethyl]methylamino) propane,/V,/V-bis(2-hydroxyethyl)-2-aminoethanesulfonic acid,/V-[tris(hydroxymethyl)methyl]-3-aminopropanesulfonic acid,/V,/V-bis (2-hydroxyethyl)glycine,/V,/V-bis(2-hydroxyethyl) taurine, diethanolamine,/V-tris(hydroxymethyl)methylglycine or a pharmaceutically acceptable salt or combination of any of the foregoing.

In some embodiments, the composition is buffered between pH 4 to 9, or between pH 5 to 8 (and all one tenth integers in between). In some embodiments, the pH is between pH 6 and 8, or between pH 6 and 7. In particular embodiments, the pH is about pH 6, 6.5, 7, 7.5 or 8.

In certain embodiments, the composition may further comprise a pH adjusting agent, such as ammonia solution, diethanolamine, meglumine, sodium citrate dihydrate, sodium hydroxide, sodium phosphate, sodium borate, sodium citrate, sodium acetate, sodium lactate, tromethamine, hydrochloric acid, trishydroxymethylamino-methane or a combination thereof.

In some embodiments, the composition further comprises a preservative. Suitable preservatives include, but are not limited to, methyl hydroxybenzoate (methylparaben), ethyl hydroxybenzoate, propyl 4-hydroxybenzoate (propylparaben), acetic acid, chlorocresol, diazolidinyl urea, dimethyl sulfoxide, imidurea, iodine/edetic acid, phenylmercuric acetate, phenylmercuric borate, phenylmercuric hydroxide, potassium sorbate, sodium hydroxide, sorbic acid, thymol, acetone sodium bisulfite, butylparaben, coconut oil, ethylparaben, sodium propionate, sporocides, vanillin, acetone sodium bisulfite, benzalkonium chloride, benzethonium chloride, benzoic acid, benzyl alcohol, benzyl benzoate, betadex sulfobutyl ether sodium, boric acid, bronopol, butylated hydroxyanisole, butylene glycol, calcium acetate, calcium chloride, calcium lactate, cetrimide, cetrimonium bromide, cetyl pyridinium chloride, chlorhexidine, chlorobutanol, chloroxylenol, chlorphenesin, cresol, hexetidine, hydrogen peroxide, iodopropynyl butylcarbamate, isopropyl alcohol, monothioglycerol, pentetic acid, phenoxyethanol, phenylethyl alcohol, phenylmercuric nitrate, phosphoric acid, potassium metabisulfite, potassium nitrate, povidone-iodine, propyl gallate, propylparaben sodium, sodium acetate, sodium benzoate, sodium metabisulfite, sodium sulfite, tetrasodium edetate, zinc oxide, dimethyl ether, lactic acid, phenol, propionic acid, sodium borate, sodium lactate, thimerosal, or a pharmaceutically acceptable salt or combination of any of the foregoing. In some embodiments, the preservative is methyl hydroxybenzoate, propyl 4-hydroxybenzoate, phenoxyethanol, chlorphenesin or a combination thereof.

In certain embodiments, the composition may further comprise an antioxidant, suitable representatives of which include acetone sodium bisulfite, alpha tocopherol, ascorbic acid, ascorbyl palmitate, butylated hydroxyanisole, butylated hydroxytoluene, citric acid monohydrate, dodecyl gallate, erythorbic acid, ethyl oleate, fumaric acid, histidine, malic acid, D-mannose, monothioglycerol, niacinamide, octyl gallate, phosphoric acid, potassium metabisulfite, propionic acid, propyl gallate, sodium ascorbate, sodium bisulfite, sodium formaldehyde sulfoxylate, sodium metabisulfite, sodium sulfite, sodium thiosulfate, sulfur dioxide, citric acid monohydrate, tartaric acid, thymol, tocopherol polyethylene glycol succinate, tocopherol, ubiquinone, idebenone, lycopene, resveratrol, niacinamide, or a pharmaceutically acceptable salt or combination of any of the foregoing.

While the composition may not comprise further active agents, the administration of other active agents concurrently with the composition is within the scope of the invention. For example, in some embodiments, the composition may be administered concurrently with one or more anti-inflammatory agents, or other anti-fungal agents. The composition may be therapeutically used after the other active agent or may be therapeutically used together with the other active agent. The composition may be administered separately, simultaneously or sequentially with the other active agent.

Exemplary anti-inflammatory agents include NSAIDs (e.g. acetylsalicylic acid (aspirin), diclofenac, diflusinal, etodolac, fenbufen, fenoprofen, flufenisal, flurbiprofen, ibuprofen, indomethacin, ketoprofen, ketorolac, meclofenamic acid, mefenamic acid, meloxicam, nabumetone, naproxen, nimesulide, nitroflurbiprofen, olsalazine, oxaprozin, phenylbutazone, piroxicam, sulfasalazine, sulindac, tolmetin, zomepirac, celecoxib, deracoxib, etoricoxib, mavacoxib, phenazone, suprofen or parecoxib), disease-modifying antirheumatic drugs (DMARDs) (e.g. methotrexate, leflunomide, sulfasalazine, hydroxychloroquinone, penicillamine, anatacept, baricitinib, cetolizumab, sarilumab, tocilizumab or tofacitinib), prednisone, methylprednisolone, dexamethasone, hydrocortisone, budesonide, mometasone, prednisolone, etanercept, golimumab, infliximab, adalimumab, anakinra, rituximab, natalizumab, abatacept, betamethasone, fluocinolone and triamcinolone.

Suitable anti-fungals include, but are not limited to, anidulafungin, caspofungin, clotrimazole, fluconazole, flucytosine, griseofulvin, griseofulvin microsize, griseofulvin ultramicrosize, isavuconazonium, itraconazole, ketoconazole, micafungin, miconazole, nystatin, posaconazole, terbinafine, voriconazole, fosfluconazole, isavuconazole, candicidin, hamycin, natamycin, bifonazole, butoconazole, econazole, fenticonazole, isoconazole, luliconazole, omoconazole, oxiconazole, sertaconazole, sulconazole, tioconazole, albaconazole, efinaconazole, terconazole, abafungin, amofolfin, butenafine, naftifine, ciclopirox, tolnafate, orotomide, oteseconazole, ibrexafungerp, rezafungin, or a pharmaceutically acceptable salt or combination of any of the foregoing.

In certain embodiments, compounds structurally related to IDB, including analogues with improved aqueous solubility and pharmacokinetic properties, are used in combination with AmB. Exemplary analogues include mitoquinone and plastoquinone. These compounds may provide similar or enhanced potentiation of AmB activity and represent alternative embodiments of the invention.

In some embodiments, the disclosed combinations reduce the toxicity associated with AmB therapy, particularly nephrotoxicity, by facilitating the use of lower effective doses. In further embodiments, the use of IDB as a relatively low-cost adjunct improves the affordability and accessibility of antifungal treatment, especially in resource-limited regions.

In certain embodiments, the composition comprises AmB at a concentration no greater than 2 μg/ml, no greater than 1.5 μg/ml, no greater than 1.2 μg/ml, no greater than 1 μg/ml, or no greater than 0.5 μg/ml. In certain embodiments, the composition comprises AmB at a concentration from 0.01 to 2 μg/ml, from 0.05 to 1.5 μg/ml, from 0.1 to 1.2 μg/ml, from 0.1 to 1 μg/ml, or from 0.2 to 0.5 μg/ml.

In certain embodiments, the composition comprises IDB at a concentration of at least about 1 μg/ml, 2 μg/ml, 5 μg/ml, 10 μg/ml, 20 μg/ml, 30 μg/ml, 40 μg/ml, 50 g/ml, 60 μg/ml, 70 μg/ml, 80 μg/ml, 90 μg/ml, or 100 μg/ml. In certain embodiments, the composition comprises IDB at a concentration from 0.1 to 5000 μg/ml, from 0.5 to 4000 μg/ml, from 1 to 3000 μg/ml, from 5 to 2000 μg/ml, from 1 to 1000 μg/ml, from 1 to 500 μg/ml, from 1 to 200 μg/ml, from 1 to 100 μg/ml, from 1 to 50 μg/ml, or from 1 to 10 μg/ml.

The pharmaceutical composition can be administered through, for example, oral, rectal, bronchial, nasal, topical, buccal, sub-lingual, transdermal, vaginal, intramuscular, intraperitoneal, intravenous, intra-arterial, intracerebral, interaocular administration or in a form suitable for administration by inhalation or insufflation, including powders and liquid aerosol administration, or by sustained release systems such as semipermeable matrices of solid hydrophobic polymers containing the compound(s) of the invention.

In specific embodiments, the compositions may be formulated for systemic administration, including intravenous or oral delivery. In certain embodiments, IDB is administered via alternative delivery routes to enhance bioavailability and tissue targeting. For example, intranasal or inhalational formulations may be used for pulmonary delivery, improving drug penetration in lung tissues for the treatment of respiratory fungal infections. Such delivery methods may enhance biomembrane permeation and therapeutic effectiveness.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the pharmaceutical compositions are formulated into ointments, salves, gels, or creams as generally known in the art.

In one embodiment, the composition may be formulated in an aqueous solution for oral administration. The composition may be dissolved in suitable solutions with added suitable colorants, flavors, stabilizing and thickening agents, artificial and natural sweeteners, and the like. In addition, the composition may further be dissolved in solution containing viscous material, such as natural or synthetic gums, resins, methylcellulose, sodium carboxymethylcellulose, or other well-known suspending agents.

Oral compositions may include an inert diluent or an edible carrier. They can be enclosed in gelatin capsules or compressed into tablets. For the purpose of oral therapeutic administration, the AmB and IDB can be incorporated with excipients and used in the form of tablets, troches, or capsules. Pharmaceutically compatible binding agents, and/or adjuvant materials can be included as part of the composition, which include, but are not limited to binders (such as microcrystalline cellulose, gelatin, a sugar, polyethylene glycol, natural and synthetic gums, polyvinylpyrrolidone, pregelatinised starch, hydroxypropyl cellulose and hydroxypropyl methylcellulose); fillers (such as such as lactose, mannitol, xylitol, dextrose, sucrose, sorbitol, microcrystalline cellulose, starch, and dibasic calcium phosphate dehydrate);

disintegrating agents (such as alginic acid, Primogel, sodium starch glycolate, sodium carboxymethyl cellulose, calcium carboxymethyl cellulose, polyvinylpyrrolidone, methyl cellulose, microcrystalline cellulose, lower alkyl-substituted hydroxypropyl cellulose, starch, pregelatinised starch and sodium alginate); lubricants (such as magnesium stearate, calcium stearate, zinc stearate, sodium stearyl fumarate, and mixtures of magnesium stearate with sodium lauryl sulfate); glidants such as colloidal silicon dioxide; sweetening agenst such as sucrose or saccharin; or flavoring agents such as peppermint, methyl salicylate, or orange flavoring.

In certain embodiments, the composition is applied topically or systemically or via a combination of both. The composition may be formulated in the forms of lotion, cream, gel and the like.

In one embodiment, the composition is formulated for parenteral administration (e.g. by injection, for example bolus injection or continuous infusion). In addition, the composition may be presented in unit dose form in ampoules, pre-filled syringes, and small volume infusion or in multi-dose containers with or without an added preservative. The compositions may be in forms of suspensions, solutions, or emulsions in oily or aqueous vehicles. The composition may further contain formulation agents such as suspending, stabilizing and/or dispersing agents. In a further embodiment, the active ingredient of the composition according to the invention may be in powder form, obtained by aseptic isolation of sterile solid or by lyophilization from solution for constitution with a suitable vehicle, e.g. sterile, pyrogen-free water, before use.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor ELTM (BASF, Parsippany, N.J.) or phosphate buffered saline (PBS). In all cases, the injectable composition should be sterile and should be fluid to the extent that easy syringability exists. It must be stable under the conditions of manufacture and storage and must be preserved against the contaminating action of microorganisms such as bacteria and fungi. The proper fluidity can be maintained, for example, by the use of a coating such as lecithin, by the maintenance of the requited particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, and sodium chloride in the composition. Prolonged absorption of the injectable compositions can be brought about by including in the composition an agent which delays absorption, for example, aluminium monostearate and gelatin. In the case of sterile powders for the preparation of sterile injectable solutions, the preferred methods of preparation are, for example, vacuum drying and freeze drying techniques.

In some embodiments, the compositions are suitable for inhalation or intranasal delivery and are in the form of, for example, solutions, aerosols, dry powders, suspensions or emulsions. For example, the composition may be administered to the respiratory tract as a nasal or pulmonary inhalation aerosol or solution for a nebuliser, or as a powder for insufflation, alone or in combination with an inert carrier, such as lactose, glucose or mannitol, or with other pharmaceutically or veterinary acceptable excipients, such as beta-cyclodextrin, starch, sodium carboxymethylcellulose and the like as discussed above. The composition may be administered by, for example, a dry powder inhaler, a soft mist inhaler, a nebuliser or a metered dose inhaler.

Aerosol formulations include those in which the composition is provided in a pressurized pack with a suitable propellant such as a metered dose inhaler. Whilst the propellant may be a chlorofluorocarbon (CFC) (such as dichlorodifluoromethane, trichlorofluoromethane or 1,2-dichlorotetrafluoroethane), the propellant is more preferably a non-chlorofluorocarbon propellant such as carbon dioxide, a hydrofluoroalkane (such as 1,1,1,2-tetrafluoroethane (HFA-134a), 1,1,1,2,3,3,3-heptafluoropropane (HFA-227) or 1,1,1,2,3,3,3-heptafluoroethane (HFA-227ea)) or another suitable gas.

The composition of the subjection invention may also be administered in a controlled release formulation such as a slow release or a fast release formulation. Such controlled release formulations of the combinations of this invention may be prepared using methods well known to those skilled in the art. The method of administration will be determined by the attendant physician or other person skilled in the art after an evaluation of the subject's condition and requirements.

In one embodiment, the pharmaceutical composition is provided in a unit dosage form, wherein the composition in desired form is divided into unit doses containing appropriate quantities of the active component. The unit dosage form can be a packaged preparation, the package containing discrete quantities such as packaged tablets, capsules, and powders in vials or ampoules. Moreover, the unit dosage form can be a capsule, tablet, cachet, or lozenge itself, or it can be the appropriate number of any of these in packaged form. In a preferred embodiment, tablet or capsule forms are for oral administration and liquid form are for intravenous administration and continuous infusion.

In one embodiment, dosage units containing AmB may contain about 0.01 mg to 50 mg, about 0.05 mg to 40 mg, about 0.1 mg to 30 mg, about 0.5 mg to 20 mg, about 1 mg to 10 mg, about 1 mg to 20 mg, or about 1 mg to 5 mg.

In one embodiment, dosage units containing IDB contain about 0.01 mg to 4000 mg, about 0.01 mg to 3000 mg, about 0.01 mg to 2000 mg, about 0.01 mg to 1000 mg, about 0.01 mg to 900 mg, about 0.01 mg to 800 mg, about 0.05 mg to 700 mg, about 0.1 mg to 600 mg, about 0.1 mg to 500 mg, about 0.1 mg to 200 mg, about 0.1 mg to 100 mg, about 0.1 mg to 90 mg, about 0.1 mg to 80 mg, about 0.1 mg to 70 mg, about 0.1 mg to 60 mg, about 0.1 mg to 50 mg, about 0.1 mg to 40 mg, about 0.1 mg to 30 mg, about 0.1 mg to 20 mg, about 0.1 mg to 10 mg, about 0.5 mg to 50 mg, about 1 mg to 40 mg, about 1 mg to 20 mg, about 1 mg to 10 mg, or about 1 mg to 5 mg.

In one embodiment, dosage units containing AmB and IDB contain about 0.01 mg to 4000 mg, about 0.01 mg to 3000 mg, about 0.01 mg to 2000 mg, about 0.01 mg to 1000 mg, about 0.01 mg to 900 mg, about 0.01 mg to 800 mg, about 0.05 mg to 700 mg, about 0.1 mg to 600 mg, about 0.1 mg to 500 mg, about 0.1 mg to 200 mg, about 0.1 mg to 100 mg, about 0.1 mg to 90 mg, about 0.1 mg to 80 mg, about 0.1 mg to 70 mg, about 0.1 mg to 60 mg, about 0.1 mg to 50 mg, about 0.1 mg to 40 mg, about 0.1 mg to 30 mg, about 0.1 mg to 20 mg, about 0.1 mg to 10 mg, about 0.5 mg to 50 mg, about 1 mg to 40 mg, about 1 mg to 20 mg, about 1 mg to 10 mg, or about 1 mg to 5 mg.

In one embodiment, the subject invention provides a method for treating or inhibiting an infection, the method comprising administering to a subject in need thereof an effective amount of a combination of AmB and IDB or an analog thereof.

In certain embodiments, the infection is a fungal infection. In specific embodiments, the fungal infection is caused by a Candida spp. (e.g., C. albicans, C. auris, C. glabrata, C. parapsilosis, C. tropicals, C. krusei), Cryptococcus spp. (e.g., C. neoformans, and C. gattii), Aspergillus spp. (e.g., A. fumigatus), Malassezia spp. (e.g. Malassezia pachydermatis or Malassezia furfur), Nocardia spp., Saccharomyces spp., Microsporum spp, or Trichophyton spp.

In specific embodiments, the fungal infection can be caused by a member of the Candida spp. selected from, for example, Candida albicans, Candida auris, Candida glabrata, Candida parapsilosis, Candida dubliensis, Candida tropicalis, Candida lusitaniae, Candida pseudoguillerimondi, and Candida krusei. In a specific embodiment, the fungal infection is caused by a member of the Cryptococcus spp., for example, Cryptococcus neoformans.

In specific embodiments, the fungal infection is caused by a multidrug-resistant fungal pathogen. In a specific embodiment, the fungal infection is resistant to AmB.

In one embodiment, the subject invention provides a method for treating or inhibiting an infection, preferably, fungal infection, the method comprising co-administering to a subject in need thereof an effective amount of AmB and IDB or an analog thereof.

In one embodiment, the subject invention provides a method for treating or inhibiting an infection, preferably, fungal infection, the method comprising administering to a subject in need thereof an effective amount of a pharmaceutical composition the subject invention. in a preferred embodiment, the pharmaceutical composition comprises AmB and IDB or an analog thereof.

In specific embodiments, the subject may be a human or non-human animal afflicted with, or at risk of developing, an infection caused by pathogenic fungi, including drug-resistant strains. In certain embodiments, the subject is an immunocompromised individual, such as a patient with HIV/AIDS, a transplant recipient, or a patient undergoing chemotherapy.

In some embodiments, the composition is administered in an amount effective to achieve a therapeutic benefit, including reduction in fungal burden, inhibition of fungal growth, or eradication of the infection. The administration may be performed via oral, intravenous, topical, inhalational, or other suitable routes, depending on the condition being treated and the formulation employed. In particular embodiments, IDB is co-administered with AmB in a manner sufficient to enhance the antifungal activity of AmB and/or reduce the required dosage and associated toxicity.

In some embodiments, the infection is an infection of the ear, skin, respiratory tract (e.g. lower respiratory tract such as the lungs), blood, urinary tract, nose or nasopharynx.

In one embodiment, the subject invention provides a method for restoring or enhancing the fungicidal activity of AmB against fungal pathogens, specifically, multidrug-resistant fungal pathogens, including Candida auris, the method comprising co-administering IDB to a subject in need thereof with AmB. The combination overcomes resistance mechanisms and re-sensitizes resistant strains to AmB, thereby improving therapeutic efficacy relative to AmB monotherapy.

In certain embodiments, IDB is administered in combination with AmB in a regimen that leverages its favorable pharmacokinetic properties, including oral bioavailability and the ability to cross the blood-brain barrier. Such embodiments are particularly useful for treating systemic and central nervous system fungal infections. In some embodiments, the established safety profile of IDB enables rapid clinical translation and flexible dosing strategies.

In a further embodiment, IDB serves a dual function as both an antifungal potentiator and a cytoprotective agent. The combination therapy reduces host cell damage associated with oxidative stress and AmB-induced toxicity, thereby improving tolerability and patient outcomes while maintaining or enhancing antifungal efficacy.

In some embodiments, the combination of AmB and IDB or an analog thereof is used to treat parasitic infections for which AmB is a standard treatment, including infections caused by Leishmania species.

In one embodiment, the subject invention provides a method for treating candidemia, the method comprising administering to a subject in need thereof an effective amount of a pharmaceutical composition of the subject invention, or a combination of AmB and IDB or an analog thereof.

In one embodiment, the subject invention provides a method for treating candidemia caused by multidrug-resistant Candida species, including Candida auris, the method comprising administering to a subject in need thereof AmB in combination with IDB or an analog thereof, or a composition of the subject invention. In specific embodiments, the candidemia is resistant to AmB, azole and/or echinocandins. The combination restores or enhances susceptibility of fungal strains resistant to AmB, azoles and/or echinocandins, thereby improving therapeutic efficacy. In certain embodiments, the dose of AmB is reduced relative to monotherapy, resulting in decreased toxicity while maintaining or enhancing antifungal activity.

In one embodiment, the invention provides a method for treating infections caused by Cryptococcus neoformans, particularly in immunocompromised patients, including those with HIV. The method comprises administering to a subject in need thereof AmB in combination with IDB or an analog thereof as an alternative or adjunct to existing regimens such as AmB plus flucytosine. In some embodiments, the combination reduces dependence on flucytosine, thereby lowering treatment costs and improving accessibility in resource-limited settings. In certain embodiments, IDB enhances central nervous system penetration of the therapeutic regimen due to its ability to cross the blood-brain barrier.

In one embodiment, the subject invention provides a method for treating invasive pulmonary aspergillosis caused by Aspergillus species, including drug-resistant Aspergillus fumigatus. The method comprises administering to a subject in need thereof AmB in combination with IDB or an analog thereof, or a composition of the subject invention, to improve antifungal efficacy, particularly in immunosuppressed patients. In certain embodiments, the combination increases treatment response rates and reduces adverse effects associated with high-dose AmB therapy.

In certain embodiments, the subject invention provides a method for inhibiting the development of an infection caused by a fungi selected from, for example, Candida spp. (e.g., C. albicans, C. auris, C. glabrata, C. parapsilosis, C. tropicals, C. krusei), Cryptococcus spp. (e.g., C. neoformans, and C. gattii), Aspergillus spp. (e.g., A. fumigatus), Malassezia spp. (e.g. Malassezia pachydermatis or Malassezia furfur), ringworm such as Microsporum spp. or Trichophyton spp., the method comprising administering to a subject in need thereof a composition of the subject invention or a combination of AmB and IDB or an analog thereof.

In certain embodiments, the method for inhibiting the development of a fungal infection in a subject comprises administering to the subject a pharmaceutical composition of the subject invention.

In certain embodiments, the method of the subject invention further comprises obtaining a sample from the subject having or suspected of having an infection, specifically, fungal infection; and measuring the fungal growth in the sample before and/or after the administration of AmB and IDB, or after the administration of each of AmB and IDB.

Examples of sources of samples include but are not limited to biopsy or other in vivo or ex vivo analysis of prostate, breast, skin, muscle, facia, brain, endometrium, lung, head and neck, pancreas, small intestine, blood, liver, testes, ovaries, colon, skin, stomach, esophagus, spleen, lymph node, bone marrow, kidney, placenta, or fetus. In some aspects of the disclosure, the sample comprises a fluid sample, such as peripheral blood, lymph fluid, ascites, serous fluid, pleural effusion, sputum, cerebrospinal fluid, amniotic fluid, lacrimal fluid, stool, or urine. Samples include single cells, whole organs or any fraction of a whole organ, in any condition including in vitro, ex vivo, in vivo, post-mortem, fresh, fixed, or frozen.

In certain embodiments, the subject invention provides a method for treating biofilm comprising a fungi, such as a Candida spp. (e.g. Candida albicans, Candida auris, Candida glabrata, Candida tropicalis or Candida parapsilosis), Malassezia spp. (e.g. Malassezia pachydermatis or Malassezia furfur), ringworm such as Microsporum spp, or Trichophyton spp., Aspergillus spp, or Cryptococcus spp.

The biofilm may be present on any surface, such as a mucous membrane, skin, a wound (e.g. a wound caused by mechanical trauma, such as physical trauma caused by an accident or a burn or a surgical wound), a body membrane, an internal body surface or an external body surface of a subject such as a human or a non-human animal. In some embodiments, the surface is skin or a mucous membrane, such as a mucous membrane lining the respiratory tract, urinary tract, reproductive organ or ear including the ear canal. In some embodiments, the surface is the surface lining the ear or ear canal, for example, the external ear or middle ear.

In certain embodiments, the method comprises contacting the biofilm with an effective amount of a combination of AmB and IDB, or a composition of the subject invention. The biofilm may be contacted with the composition of the subject invention for a suitable time to disrupt or eradicate the biofilm. For example, in some embodiments, the biofilm is contacted with the composition for a period from about 10 seconds to about 24 hours, about 30 seconds to about 24 hours, about 1 minute to about 24 hours, about 30 minutes to about 24 hours, about 1.5 to about 24 hours, about 3 to about 24 hours, about 30 minutes to about 3 hours, about 30 minutes to about 1.5 hours or about 1.5 and about 3 hours. In some embodiments, the biofilm is contacted with the composition for at least about 10, 15, 20, 25, 30, 35, 40, 45, 50, 55 or 60 seconds; or about 10, 15, 20, 25, 30, 35, 40, 45, 50, 55 or 60 seconds. In some embodiments, the biofilm is contacted with the composition for at least about 30 or 45 minutes or about 1, 1.5, 2, 2.5, 3, 3.5, 4, 4.5, 5, 5.5, 6, 6.5, 7, 7.5, 8, 8.5, 9, 9.5, 10, 11, 12, 13, 14, 15, 16, 17, 18, 19, 20, 21, 22, 23 or 24 hours.

In certain embodiments, the composition, once administered, provides a plasma concentration of AmB no greater than 2 μg/ml, no greater than 1.5 μg/ml, no greater than 1.2 ug/ml, no greater than 1 μg/ml, or no greater than 0.5 μg/ml. In certain embodiments, the composition, once administered, provides a plasma concentration of AmB ranging from 0.01 to 2 μg/ml, from 0.05 to 1.5 μg/ml, from 0.1 to 1.2 μg/ml, from 0.1 to 1 μg/ml, or from 0.2 to 0.5 μg/ml.

In certain embodiments, the composition, once administered, provides a plasma concentration of IDB of at least about 1 μg/ml, 2 μg/ml, 5 μg/ml, 10 μg/ml, or 20 μg/ml. In certain embodiments, the composition, once administered, provides a plasma concentration of IDB ranging from 0.1 to 500 μg/ml, from 0.5 to 400 μg/ml, from 1 to 300 μg/ml, from 5 to 200 μg/ml, from 1 to 100 μg/ml, from 1 to 50 μg/ml, from 1 to 20 μg/ml, or from 1 to 10 μg/ml. Idebenone exhibits an exemplary safety profile, permitting administration of up to 4 grams daily.

The compositions of the present invention can be administered to the subject being treated by standard routes, including the local, oral, buccal, bronchial, ophthalmic, sub-lingual, nasal, topical, intratumoural, transdermal, intra-articular, parenteral (e.g., intravenous, intraperitoneal, intradermal, subcutaneous or intramuscular), intracranial, intracerebral, intraspinal, intravaginal, intrauterine, or rectal route. Administration may be also by way of other carriers or vehicles such as patches, micelles, liposomes, vesicles, implants (e.g., microimplants), synthetic polymers, microspheres, nanoparticles, and the like. Depending on the condition being treated, one route may be preferred over others, which can be determined by those skilled in the art.

In certain embodiments, the subject invention provides a method of conferring susceptibility to a resistant fungi, the method comprising contacting the fungi with a pharmaceutical composition of the subject invention or a combination of AmB and IDB or an analog thereof.

The fungi may be contacted with the composition for a suitable time to confer susceptibility. For example, in some embodiments, the fungi is contacted with the composition for a period from about 10 seconds to about 24 hours, about 30 seconds to about 24 hours, about 1 minute to about 24 hours, about 30 minutes to about 24 hours, about 1.5 to about 24 hours, about 3 to about 24 hours, about 30 minutes to about 3 hours, about 30 minutes to about 1.5 hours or about 1.5 and about 3 hours. In some embodiments, the fungi is contacted with the composition for at least about 10, 15, 20, 25, 30, 35, 40, 45, 50, 55 or 60 seconds; or about 10, 15, 20, 25, 30, 35, 40, 45, 50, 55 or 60 seconds. In some embodiments, the fungi is contacted with the composition for at least about 30 or 45 minutes or about 1, 1.5, 2, 2.5, 3, 3.5, 4, 4.5, 5, 5.5, 6, 6.5, 7, 7.5, 8, 8.5, 9, 9.5, 10, 11, 12, 13, 14, 15, 16, 17, 18, 19, 20, 21, 22, 23 or 24 hours.

Contacting a fungi is understood herein as providing an agent to a fungi, e.g., a fungi to be treated in culture, ex vivo, or in an animal, such that the agent can interact with the fungi, and have an effect on the cell. The agent can be delivered to the fungi directly (e.g., by addition of the agent to culture medium or by injection into the cell or tissue of interest), or by delivery to the organism by, e.g., a topical or parenteral route of administration for delivery to the fungi by vascular, lymphatic, or other means.

The terms “treatment” or any grammatical variation thereof (e.g., treat, treating, etc.), as used herein, includes but is not limited to, the application or administration to a subject (or application or administration to a cell or tissue from a subject) with the purpose of slowing, stabilizing, curing, healing, alleviating, relieving, remedying, less worsening, ameliorating, or improving the disease or condition. The term “treating” refers to any indication of success in the treatment or amelioration of a pathology or condition, including any objective or subjective parameter such as abatement; remission; lessening of the rate of worsening; lessening severity of the disease; stabilization, diminishing of symptoms or making the pathology or condition more tolerable to the subject; or improving a subject's physical well-being. In certain embodiments, treatment can refer to a 10%, 20%, 30%, 40%, 50%, 60%, 70%, 80%, 90%, or 100% reduction in the severity of an established fungal infection or symptom of the fungal infection.

As used herein, the term “subject” refers to an animal, needing the benefits provided by a therapeutic compound. The animal may be for example, humans, pigs, horses, goats, cats, mice, rats, dogs, apes, fish, chimpanzees, orangutans, guinea pigs, hamsters, cows, sheep, birds, chickens, as well as any other vertebrate or invertebrate. The preferred subject in the context of this invention is a human. The subject can be of any age or stage of development, including infant, toddler, adolescent, teenager, adult, or senior.

As used herein, the terms “therapeutically-effective amount,” “therapeutically-effective dose,” “effective amount,” and “effective dose” are used to refer to an amount or dose of a compound or composition that, when administered to a subject, is capable of treating or improving a condition, disease, or disorder in a subject or that is capable of providing enhancement in health or function to an organ, tissue, or body system. In other words, when administered to a subject, the amount is “therapeutically effective.” The actual amount will vary depending on a number of factors including, but not limited to, the particular condition, disease, or disorder being treated or improved; the severity of the condition; the particular organ, tissue, or body system of which enhancement in health or function is desired; the weight, height, age, and health of the patient; and the route of administration.

A “synergistically effective” amount or any grammatical variation thereof of AmB and IDB is an amount which, when combined, produces a greater effect than when either of AmB and IDB are used alone. The term “greater effect” encompasses not only a reduction in symptoms of the infection to be treated, but also reduced side effects, improved tolerability, improved subject compliance, improved efficacy, or any other improved clinical outcome.

The terms “co-administration,” “administered in combination with,” and their grammatical equivalents encompass administration of two or more agents to a subject so that both agents are present in the subject at the same time. Co-administration includes simultaneous administration in separate compositions, administration at different times in separate compositions, or administration in a composition in which both agents are present. Co-administered agents may be in the same formulation. Co-administered agents may also be in different formulations.

As used herein, the singular forms “a,” “an,” and “the” are intended to include the plural forms as well, unless the context clearly indicates otherwise. Furthermore, to the extent that the terms “including,” “includes,” “having,” “has,” “with,” or variants thereof are used in either the detailed description and/or the claims, such terms are intended to be inclusive in a manner similar to the term “comprising.” The transitional terms/phrases (and any grammatical variations thereof) “comprising,” “comprises,” and “comprise” can be used interchangeably; “consisting essentially of,” and “consists essentially of” can be used interchangeably; and “consisting,” and “consists” can be used interchangeably.

The transitional term “comprising,” “comprises,” or “comprise” is inclusive or open-ended and does not exclude additional, unrecited elements or method steps. By contrast, the transitional phrase “consisting of” excludes any element, step, or ingredient not specified in the claim. The phrases “consisting” or “consists essentially of” indicate that the claim encompasses embodiments containing the specified materials or steps and those that do not materially affect the basic and novel characteristic(s) of the claim. Use of the term “comprising” contemplates other embodiments that “consist” or “consisting essentially of” the recited component(s).

The term “about” or “approximately” means within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 0 to 10%, 0 to 5%, or up to 1% of a given value. Where particular values are described in the application and claims, unless otherwise stated the term “about” meaning within an acceptable error range for the particular value should be assumed. In the context of compositions containing amounts of concentrations of ingredients where the term “about” is used, these values include a variation (error range) of 0-10% around the value (X+10%).

Ranges are stated in shorthand to avoid having to set out at length and describe each and every value within the range. Any appropriate value within the range can be selected, where appropriate, as the upper value, lower value, or the terminus of the range. For example, a range of 0.1-1.0 represents the terminal values of 0.1 and 1.0, as well as the intermediate values of 0.2, 0.3, 0.4, 0.5, 0.6, 0.7, 0.8, 0.9, and all intermediate ranges encompassed within 0.1-1.0, such as 0.2-0.5, 0.2-0.8, 0.7-1.0, etc. Values having at least two significant digits within a range are envisioned, for example, a range of 5-10 indicates all the values between 5.0 and 10.0 as well as between 5.00 and 10.00 including the terminal values. When ranges are used herein, combinations and subcombinations of ranges (e.g., subranges within the disclosed range) and specific embodiments therein are explicitly included.

The term “and/or” as used herein refers to and encompasses any and all possible combinations of one or more of the associated listed items. For example, the phrase “A, B, and/or C” includes A alone, B alone, C alone, the combination of A and B, the combination of A and C, the combination of B and C, and the combination of A, B, and C. Also, the term “or” is used in its inclusive sense (and not in its exclusive sense) so that when used, for example, to connect a list of items, the term “or” means one, some, or all of the items in the list. Conjunctive language such as the phrase “at least one of X, Y, and Z,” unless specifically stated otherwise, is otherwise understood with the context as used in general to convey that an item, term, etc. may be either X, Y, or Z; X and Y; X and Z; Y and Z; or X, Y, and Z (i.e., any combination of X, Y, and Z).

Throughout this application, where publications are referenced, the disclosures of these publications in their entireties are hereby referred to in this application to more fully describe the state of the art to which this invention pertains.

Unless otherwise defined, all terms of art, notations and other scientific terms or terminology used herein are intended to have the meanings commonly understood by those of skill in the art to which this invention pertains. In some cases, terms with commonly understood meanings are defined herein for clarity and/or for ready reference, and the inclusion of such definitions herein should not necessarily be construed to represent a substantial difference over what is generally understood in the art. It will be further understood that terms, such as those defined in commonly used dictionaries, should be interpreted as having a meaning that is consistent with their meaning in the context of the relevant art and/or as otherwise defined herein.

### EXAMPLES

### Materials and Methods

### Fungal Isolates, Media, Chemicals, and Tested Drugs

A total of 43 fungal isolates were used in this study. 25 C. auris isolates, 3 C. albicans, 3 C. glabrata, 2 C. parapsilosis, 2 C. tropicals, 2 C. krusei, 2 C. neoformans, 2 C. gattii and 2 A. fumigatus strains were obtained from the U.S. Centers for Disease Control and Prevention (Atlanta, GA, USA), BEI Resources (Manassas, VA, USA), and the Westerdijk Fungal Biodiversity Institute (Utrecht, The Netherlands). Media and tested drugs were sourced as follows: RPMI 1640 (Gibco, Grand, Island, NY, USA); 3-(N-Morpholino) propane sulfonic acid (MOPS) (Fisher Bioreagents, Fairlawn, NJ); phosphate buffered saline (PBS) (Corning, Manassas, VA, USA); Yeast extract Peptone Dextrose agar and broth (YPD, Becton, Dickinson, and Company, Sparks, MD, USA); amphotericin B (Apexbio, Houston, USA); and idebenone (TCI America, Portland, OR, USA).

### Screening, In Vitro MICs, and Checkerboard Assays

Idebenone was identified as a potent enhancer of amphotericin B's antifungal activity from a screen against C. auris AR0390 in the presence of a subinhibitory concentration of amphotericin B. The MICs of amphotericin B and idebenone against fungal isolates were identified according to CLSI M27-A3. The endpoints were identified as 100% inhibition of fungal growth for amphotericin B and idebenone. Results were taken after 24 hours for Candida, 72 hours for Cryptococcus, and 48 hours for Aspergillus species. The in vitro interaction between amphotericin B and idebenone was evaluated against 25 C. auris, 3 C. albicans, 3 C. glabrata, 2 C. neoformans, 2 C. gattii and 2 A. fumigatus isolates using the microdilution checkerboard assay. The ΣFICI was determined and interpreted according to the following: ΣFICI ≤0.5 was considered as synergism (SYN), ΣFICI from >0.5 to $4 was considered as indifference (IND), and ΣFICI >4 was considered as antagonism (ANT).

### Time-Kill Assay

To determine the change in the growth kinetics of C. auris in response to amphotericin B/idebenone combination treatment, a time-kill assay was utilized. In brief, C. auris AR0390 was grown to log phase in YPD broth and then diluted to 1×10⁴ CFU/mL in RPMI 1640 MOPS medium. C. auris cells were then treated with amphotericin B (1 μg/mL), idebenone (8 ug/mL), or a combination of amphotericin B and idebenone. C. auris cells were subsequently incubated at 35° C. for 24 hours. At specific time points (0, 2, 4, 6, 12, and 24 hours), aliquots from the control and treated C. auris cells were serially diluted, plated on YPD agar plates, and the CFUs were counted after 24 hours.

In Vivo Efficacy on Caenorhabditis elegans Infection Model

To evaluate the in vivo efficacy of the amphotericin B/idebenone combination, the C. elegans infection model was utilized. Briefly, C. elegans (strain AU37) were synchronized using the alkaline bleach method, and the larva stage was collected on day 4 (L4) and infected with ~1×10⁸ log phase C. auris AR0390. After 3 hours of incubation at room temperature, worms were washed twice with PBS and divided into groups in M9 buffer plus 20% RPMI 1640. The worms were divided into groups and subsequently treated with amphotericin B (0.5 μg/mL), idebenone (10 μg/mL), or a combination of amphotericin B and idebenone at the same concentrations. One group was used as an untreated control. The treatments continued for 24 hours at room temperature before the worms were collected, washed with PBS, and grounded using silicon carbide beads for 10 minutes. The homogenates were then serially diluted and plated onto YPD agar plates containing chloramphenicol (75 μg/ml) and counted after 24 hours. C. elegans results were analyzed through a one-way ANOVA with post hoc Dunnett's test for multiple comparisons using GraphPad Prism version 8.

### Example 1—Identification of Idebenone as a Potent Enhancer of Amphotericin B Against C. auris

To identify a drug capable of reverting resistance of amphotericin B and potentiate its antifungal activity, ~3400 FDA-approved drugs and clinical compounds (MCE HY-L066) were screened and the semisynthetic product idebenone was identified as a potent enhancer of amphotericin B's antifungal activity.

Combination therapy is a strategic approach as it employs a potentiator drug to enhance the effectiveness of amphotericin B, lowers the required dose, reduces toxicity, and improves efficacy.

### Example 2—Idebenone Restores the Fungicidal Activity of AmB Against Resistant C. auris

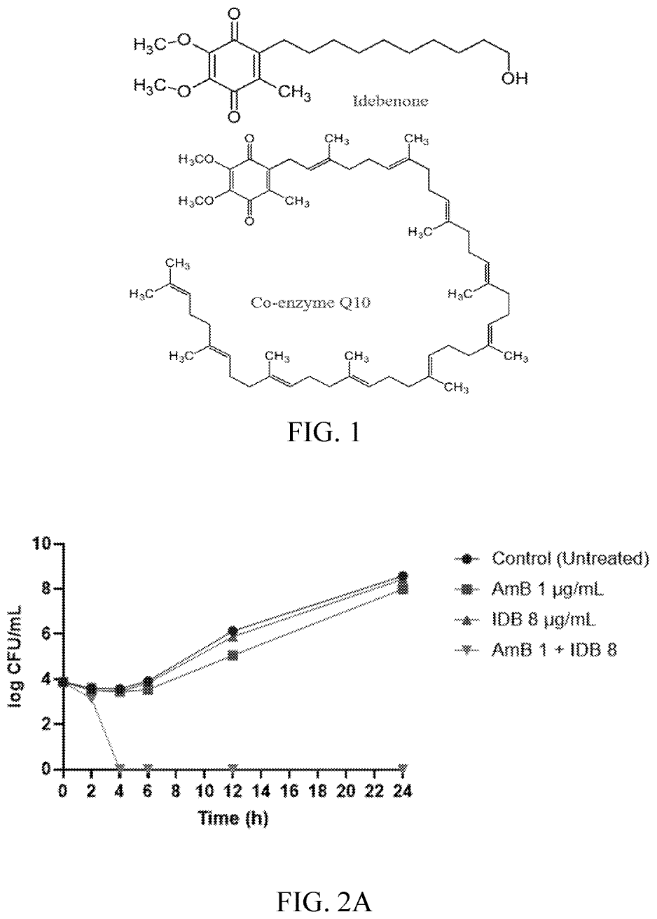

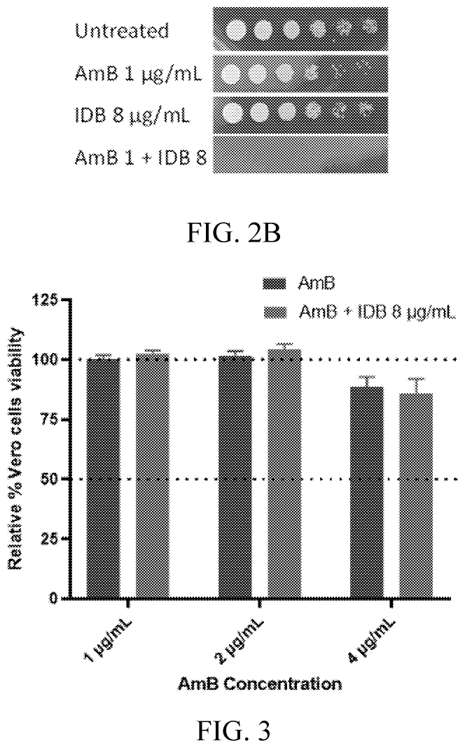

Idebenone was identified as a potent enhancer of AmB against the AmB-resistant C. auris AR0390 (South Asia, clade I, MIC of AmB=2 μg/mL). The killing kinetics of Idebenone (IDB)/AmB combination was then investigated. IDB (8 μg/mL), by itself, did not exhibit inhibitory activity against C. auris (FIG. 2A). Also, AmB, at sub-inhibitory concentration (0.5x MIC), did not exhibit fungicidal activity (FIG. 2A). When IDB was added to AmB, it restored its fungicidal activity resulting in a rapid and complete elimination of C. auris after 4 hours (FIG. 2A). Scanning images of CFUs of C. auris on YPD agar after 24 hours incubation with various treatments were shown in FIG. 2B. This further confirms the efficacy of the combination therapy against amphotericin B-resistant C. auris.

### Example 3—Antifungal susceptibility testing

The minimum inhibitory concentrations (MICs) were evaluated for amphotericin B and idebenone against several fungal species (Table 1) in accordance with CLSI guidelines. Of the tested C. auris isolates, 13/25 (52%) showed resistance to amphotericin B with MIC=2 μg/mL. Additionally, one Candida glabrata, two Candida krusei, and one Aspergillus fumigatus isolates showed elevated MICs for amphotericin B (2 μg/mL). Idebenone alone did not exhibit antifungal activity against all Candida and Aspergillus species that were tested. However, against Cryptococcus isolates, idebenone exhibited weak antifungal activity against both C. gattii and C. neoformans (MIC=64 μg/mL).

TABLE 1
In vitro interaction between amphotericin B and
idebenone against 25 C. auris isolates.

| | MIC (μg/ml) | | | | | |
|---|---|---|---|---|---|---|
| | Alone | | Combined | | | Interpre- |
| Fungal isolates | AmB | IDB | AmB | IDB | ΣFICI | tation |
| C. auris AR0381 | 1 | >128 | 0.125 | 8 | 0.16 | SYN |
| C. auris AR0382 | 1 | >128 | 0.25 | 4 | 0.27 | SYN |
| C. auris AR0383 | 1 | >128 | 0.25 | 4 | 0.27 | SYN |
| C. auris AR0384 | 1 | >128 | 0.125 | 4 | 0.14 | SYN |
| C. auris AR0385 | 2 | >128 | 0.5 | 8 | 0.28 | SYN |
| C. auris AR0386 | 2 | >128 | 0.5 | 8 | 0.28 | SYN |
| C. auris AR0387 | 1 | >128 | 0.25 | 8 | 0.28 | SYN |
| C. auris AR0388 | 2 | >128 | 0.5 | 8 | 0.28 | SYN |
| C. auris AR0389 | 2 | >128 | 0.5 | 8 | 0.28 | SYN |
| C. auris AR0390 | 2 | >128 | 0.5 | 4 | 0.27 | SYN |
| C. auris CBS 10913 | 0.5 | >128 | 0.125 | 4 | 0.27 | SYN |
| C. auris CBS 12372 | 1 | >128 | 0.25 | 4 | 0.27 | SYN |
| C. auris CBS 12373 | 1 | >128 | 0.25 | 4 | 0.27 | SYN |
| C. auris CBS 12766 | 2 | >128 | 0.5 | 8 | 0.28 | SYN |
| C. auris CBS 12768 | 2 | >128 | 0.5 | 4 | 0.27 | SYN |
| C. auris CBS 12770 | 2 | >128 | 0.5 | 8 | 0.28 | SYN |
| C. auris CBS 12771 | 2 | >128 | 0.5 | 8 | 0.28 | SYN |
| C. auris CBS 12772 | 2 | >128 | 0.5 | 8 | 0.28 | SYN |
| C. auris CBS 12773 | 2 | >128 | 0.5 | 8 | 0.28 | SYN |
| C. auris CBS 12774 | 2 | >128 | 0.5 | 8 | 0.28 | SYN |
| C. auris AR1100 | 0.5 | 128 | 0.125 | 8 | 0.31 | SYN |
| C. auris AR1101 | 0.5 | >128 | 0.125 | 4 | 0.27 | SYN |
| C. auris AR1102 | 1 | >128 | 0.25 | 4 | 0.27 | SYN |
| C. auris AR1103 | 1 | >128 | 0.25 | 8 | 0.28 | SYN |
| C. auris AR1104 | 2 | >128 | 0.25 | 8 | 0.16 | SYN |

### Example 4—Idebenone Works Synergistically with AmB Against Important Fungal Species

The interactions between amphotericin B and idebenone were investigated in vitro against 25 clinical isolates of C. auris using the standard checkerboard assay. The combination of idebenone and amphotericin B showed a synergistic relationship against all tested isolates representing the 5 clades of C. auris, producing fractional inhibitory concentration index (ΣFICI) values between 0.14 and 0.31 (Table 1).

The combination of amphotericin B/idebenone was also investigated against other Candida species, including C. albicans, C. parapsilosis, C. glabrata, C. krusei, and C. tropicals. These medically important species are responsible for approximately 90% of Candida infection cases. Furthermore, the synergistic effect of amphotericin B and idebenone has been observed across various fungal species, including Cryptococcus and Aspergillus species (Table 2). This highlights the broad applicability of this combination therapy beyond targeting C. auris.

TABLE 2
In vitro interaction between amphotericin B and idebenone against different
species of Candida, Cryptococcus, and Aspergillus.

| | MIC (μg/ml) | | | | | |
|---|---|---|---|---|---|---|
| | Alone | | Combined | | | |
| Fungal isolates | AmB | IDB | AmB | IDB | ΣFICI | Interpretation |
| C. albicans NR-29448 | 1 | >128 | 0.25 | 4 | 0.27 | SYN |
| C. albicans ATCC 573 | 1 | >128 | 0.25 | 4 | 0.27 | SYN |
| C. albicans ATCC 64124 | 1 | >128 | 0.25 | 8 | 0.28 | SYN |
| C. glabrata AR0328 | 1 | >128 | 0.25 | 8 | 0.28 | SYN |
| C. glabrata AR0329 | 1 | >128 | 0.25 | 8 | 0.28 | SYN |
| C. glabrata AR0330 | 2 | >128 | 0.5 | 4 | 0.27 | SYN |
| C. parapsilosis ATCC 22019 | 0.5 | >128 | 0.125 | 4 | 0.27 | SYN |
| C. parapsilosis CAB 502638 | 0.5 | >128 | 0.125 | 8 | 0.28 | SYN |
| C. tropicalis ATCC 1369 | 1 | >128 | 0.25 | 8 | 0.28 | SYN |
| C. tropicalis ATCC 13803 | 1 | >128 | 0.25 | 8 | 0.28 | SYN |
| C. krusei ATCC 34135 | 2 | >128 | 0.25 | 4 | 0.14 | SYN |
| C. krusei CAB 396420 | 2 | >128 | 0.5 | 8 | 0.28 | SYN |
| C. gattii NR-43210 | 1 | 64 | 0.25 | 4 | 0.31 | SYN |
| C. gattii NR-43209 | 0.5 | 64 | 0.125 | 8 | 0.38 | SYN |
| C. neoformans NR-41291 | 1 | 64 | 0.25 | 8 | 0.38 | SYN |
| C. neoformans NR-41296 | 1 | 64 | 0.125 | 8 | 0.25 | SYN |
| A. fumigatus AR0731 | 1 | >128 | 0.25 | 4 | 0.27 | SYN |
| A. fumigatus AR0733 | 2 | >128 | 0.5 | 8 | 0.28 | SYN |

C. neoformans, a leading pathogen on the WHO Fungal Priority Pathogens List, poses a significant threat, especially to immunosuppressed and HIV-patients, causing nearly 20% of HIV-related mortality globally. Amphotericin B combined with flucytosine is the most effective and recommended agents for treatment of Cryptococcus infections. However, limitations in flucytosine accessibility, primarily due to cost, hinder treatment availability, particularly in high-burden regions like Africa, South America, and Asia. The present invention indicated a synergistic relationship between idebenone and amphotericin B against Cryptococcus gattiilneoformans. Furthermore, idebenone's ability to traverse the blood-brain barrier upon oral intake, renders it a promising candidate for the treatment of cryptococcal meningitis.

Invasive pulmonary aspergillosis poses a life-threatening risk to immunosuppressed patients. Amphotericin B is a major antifungal drug used in its management, but responses, particularly in severely immunosuppressed patients, are suboptimal, with efficacy below 40% in addition to multiple serious side effects. A significant correlation has been identified between therapeutic failure and mortality in cases where amphotericin B was employed for treating infections caused by Aspergillus isolates with MICs of >2 μg/mL.

The demonstrated synergistic interaction between AmB and IDB across multiple species supports the use of the combination of AmB and IDB as a general antifungal strategy, not limited to a single pathogen.

### Example 5—Idebenone Did not Increase the Cytotoxicity of AmB Against Vero Cells

The addition of IDB did not affect the cytotoxicity of AmB against monkey kidney (Vero) cells. No significant difference was observed between AmB and AmB/IDB-treated cells (FIG. 3). This result indicates that enhanced antifungal activity of AmB by addition of IDB is not due to cell toxicity.

### Example 6—the AmB/IDB Combination Restored AmB Activity and Significantly Reduced C. auris Burden in Infected C. elegans In Vivo Model

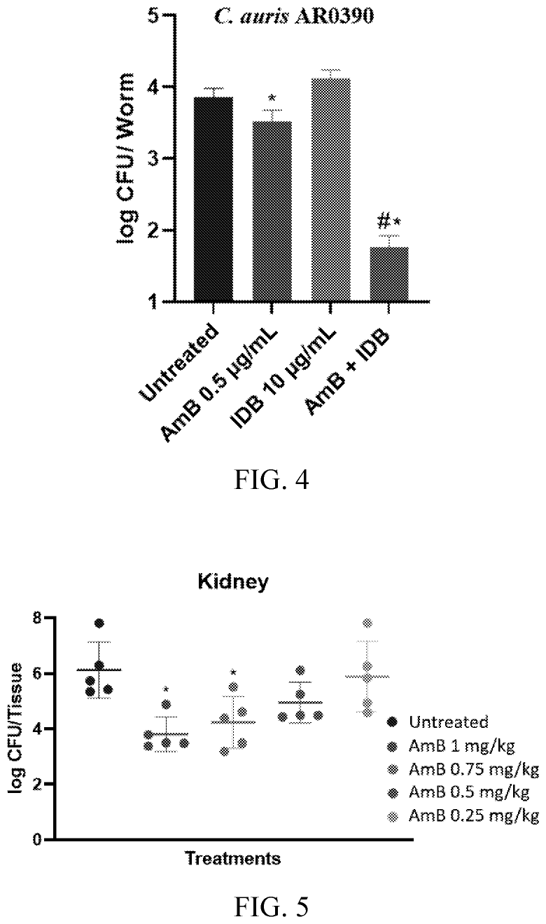

To further corroborate the activity of the AmB/IDB combination, the combination's in vivo efficacy was evaluated using a C. elegans infection model. Synchronized C. elegans worms were infected with C. auris and then treated with amphotericin B (0.5 μg/mL), idebenone (10 μg/mL), or combination of both drugs. AmB alone reduced the burden of C. auris by 0.33 log 10 CFU, while the combination reduced the burden of C. auris by 2.09-log 10 CFU compared to the untreated control and by 1.76-log 10 CFU compared to the AmB-treated group (FIG. 4).

Tolerability is one of the major concerns in the development of novel antifungal therapeutic regimens, particularly when amphotericin B is included. To mitigate the toxicity associated with amphotericin, various formulations, including liposomal, colloidal, and lipid complex forms, have been developed. However, these formulations have not completely alleviated the associated side effects. In this context, idebenone appears to be a promising compound because it demonstrates an exemplary safety profile, permitting administration of up to 4 grams daily, with no observed toxicity in human subjects. Furthermore, idebenone appears to possess cytoprotective activity in various eukaryotic cells, including human renal cells.

Altogether, these data illustrate the remarkable potential of combination therapy to enhance the effectiveness of amphotericin B and reduce potential toxicity. Idebenone's advantageous pharmacokinetic properties, including oral bioavailability and its ability to cross the blood-brain barrier, make it advantageous for clinical use in combination therapy. Finally, the relatively low cost of production of idebenone compared to other novel antifungal agents makes it an attractive option for combination therapy, especially in regions with high disease burden and limited access to more expensive drugs, like flucytosine.

### Example 7—Interactions Between IDB and AmB Against a Broader Range of Fungal Pathogens

IDB with AmB against C. auris isolates and against a panel of isolates, including C. albicans, C. parapsilosis, C. glabrata, C. krusei, C. gattii, C. neoformans, and A. fumigatus (Tables 1 and 2) were tested. A further larger panel of fungal clinical isolates (N>200) can also be tested using the AmB/IDB combination to improve rigor and reproducibility.

The interaction between IDB and AmB can be assessed using the standard microdilution checkerboard assays. The ΣFICI can be used to assess the interactions between the tested drug combinations. To study the killing kinetics of the combination, a time-kill assay and spotting assay can be performed.

In addition, whether the potential therapeutic application of AmB/IDB can be expanded beyond just inhibition of planktonic Candida. Mature-phase C. auris and C. albicans biofilms can be examined. Biofilms are exposed to a range of AmB/IDB combinations. The metabolic activity (XTT) reduction assay can be used to determine the sessile MICs (SMIC50 and SMIC80), which represent the concentration of drug where the absorbance of the biofilm decreases by 50% or by 80%, respectively, compared with the biofilm formed by the same strain in the absence of the drug.

Further evaluation can be done for the impact of the combination on hyphae formation, a key virulence factor expressed by certain Candida species that contributes to the transition of yeast to branched filamentous cells. C. albicans is diluted in RPMI-1640 medium supplemented with MOPS (at ~1×10⁵ CFU/mL) and then treated with sub-inhibitory concentrations of AmB alone (at 0.5×, 0.125× and 0.06×MIC) or combined with different concentrations of IDB in a 96-well tissue culture plates. Candida cells are incubated at 35° C. for 3 h. The morphological changes and hyphae formation can be examined using Olympus BX41 Phase Contrast & Darkfield Microscope (40× objective).

### Example 8—Mutation and Mapping Cryptococcus Isolate Resistant to the IDB

IDB does not exhibit direct antifungal activity (MIC >128 μg/mL) against all tested strains, except for Cryptococcus species (MIC=64 μg/mL). As a haploid organism possessing thoroughly studied genetics, C. neoformans stands out as an ideal model for unraveling antifungal drug targets. In vitro frequency of resistance studies can be conducted on Cryptococcus to isolate mutants and identify potential targets or resistance mechanisms.

Mutants of C. neoformans that are resistant to IDB can be generated in vitro using established methods: (a) Solid media selection of drug-resistant spontaneous mutants and (b) Liquid passaging and evolution of resistance in the presence of drug. Mutants from methods (a) and (b) can be selected and confirmed for its stability. Cryptococcus genomic DNA is isolated and sequenced and individual high-confidence SNPs can be identified. Genetic variant annotation and effect prediction software (snpEff and snpSift) are utilized to predict the impact of a specific mutation on protein function. Selected SNPs can be confirmed independently by PCR amplifying and sequencing of the PCR product. Potential target proteins can be expressed and purified using affinity-tag purification to allow for follow-up assays and target confirmation. Depending on the target, protein biochemical assays can be designed to measure the effect of the drugs on the appropriate biochemical activity. Once the protein is purified, IDB can be tested for binding assays using thermal shift, surface plasmon resonance (SPR), biolayer interferometry (BLI), and/or microscale thermophoresis (MST). If binding is confirmed, the same experiments can be performed in a dose-response manner to determine binding affinity to the target.

### Example 9—Assessing Toxicity of AmB/IDB Combination In Vitro and In Vivo

This comparative analysis of the novel combination (AmB/IDB) versus AmB alone focuses on human cell lines representing critical side effects associated with AmB, including nephrotoxicity (using primary human Renal Proximal Tubule Epithelial Cells; RPTEC), hepatotoxicity (using the human Hepatoma cell line; HepG2), and hemolysis (using human Red Blood Cells; RBCs).

To evaluate the in vitro safety profile, AmB/IDB combination against three types of human cells, RPTECs, HepG2, and human RBCs is tested. For RPTECS and HepG2, cells are incubated in proper media with different concentrations of AmB and IDB (alone and in combination) in 96-well tissue culture plates at 37° C. with 5% CO₂ for 24 h. DMSO-treated cells serve as a negative control for comparison. The viability of the cells can be evaluated using MTS assay. Acute blood hemolysis is recognized as a serious toxicity that can occur following AmB treatment. For this reason, freshly obtained 1 mL RBCs from human blood is diluted with 4 mL PBS and added to different concentrations of AmB (1-128 μg/mL), both alone and in combination with IDB. 2% sodium dodecyl sulfate (SDS) serves as a positive control whereas 1% DMSO and/or PBS serves as negative controls. After 45 min incubation period in a 96-well plates, the plate is centrifuged at 3000 rpm for 5 min and the absorbance of supernatant is measured at 540 nm (using a BioTek Synergy H1 microplate reader) to assess the degree of hemolysis.

IDB exhibited minimal toxicity in repeat dose studies across various species. Notably, IDB's maximum tolerated dose (MTD) is exceptionally high, reaching 2000 mg/kg in mice and 2500 mg/kg in rats. In this sub-aim, the objective is to investigate the toxicity of the AmB/IDB combination in C57BL/6 mice. Such inbred mouse strain has heightened sensitivity to the toxic effects of AmB.

MTD studies are performed in C57BL/6 mice in 2 phases. In Phase A, the dose level of AmB and AmB/IDB increases until the MTD is determined (n=6 animals/dose using 3 males and 3 females). The MTD is a dose that produces neither mortality nor more than a 10% decrement in body weight nor clinical signs of toxicity or a significant change in renal and/or hepatic function in the survivors. In Phase B, animals are dosed daily for 14 days at fractions of the single dose MTD to estimate a repeat dose MTD (n=10 animals/dose, 5 males and 5 females, with control, low, mid, and high dose determined from the phase A studies). During both phases, mice are observed daily for body weight change, clinical signs of abnormality, and renal and hepatic function. At termination of Phase B, samples for clinical chemistry, hematology, and histopathology can be collected.

### Example 10: In Vivo Efficacy of IDB with AmB Against C. auris

Patients with candidemia resistant to both azole and echinocandins face very limited treatment options. The sole recourse for these patients is AmB, which can be toxic, especially for individuals who are already in a compromised health condition. Moreover, approximately 30% of the circulating C. auris strains are resistant to AmB. Therefore, co-drugs such as IDB capable of enhancing the antifungal activity of AmB and reducing the dose, hold paramount clinical significance. We successfully developed a C. auris animal model and optimized the dose of AmB to be used against multidrug resistant strain of C. auris AR0390 (South Asia, clade I) (FIG. 5).

### Example 11: Validation of Efficacy In Vivo Against Cryptococcus

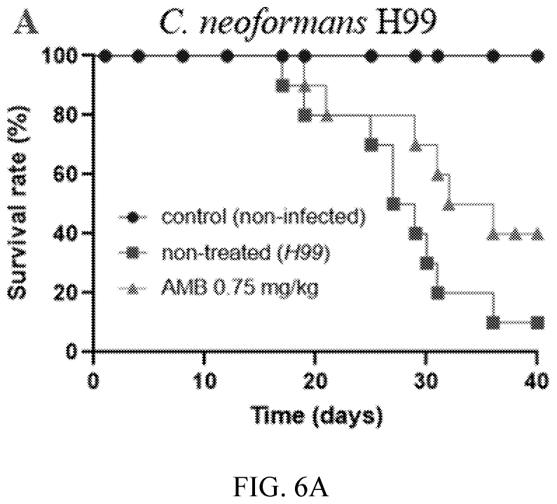

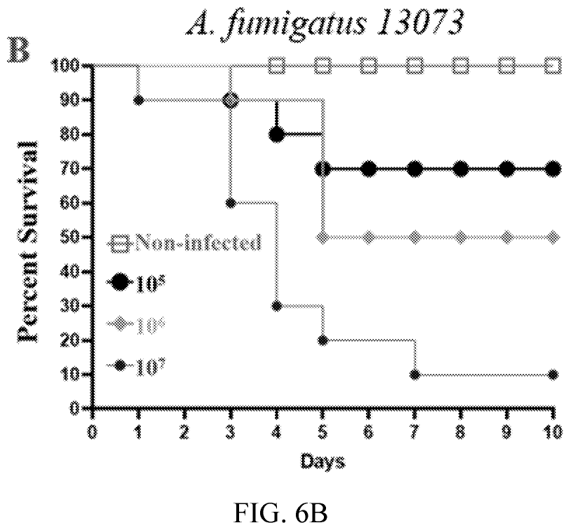

To evaluate the clinical potential of IDB for treating cryptococcal meningitis, the combination of AmB and IDB was used in established mouse model of cryptococcosis. The model is already established (FIG. 6A).

### Example 12: In Vivo Efficacy of IDB Against Aspergillus

The optimal inoculum of A. fumigatus was established, which would cause a LD₉₀ (lethal dose that kills 90%) in mice for 4 strains (FIG. 6B, Data for strain 1307 is shown). The MED₅₀ (the minimum effective dose of AmB that results in survival of 50% or less for each strain) can also be determined.

Different formulations or delivery methods for IDB can also be investigated, such as intranasal delivery, which has been shown to enhance biomembrane permeation and bioavailability. Additionally, other analogues of IDB characterized by enhanced water-solubility and improved pharmacokinetic properties, such as mitoquinone and plastoquinone can be tested as well. These analogues may offer similar or even superior efficacy in combination with AmB, providing an alternative strategy for enhancing antifungal activity.

These data offer valuable insights into the therapeutic efficacy of IDB against life-threatening fungal infections. The findings across Candida, Cryptococcus, and Aspergillus species have broad relevance to other clinically significant fungal and parasitic pathogens, including Leishmania, for which AmB is a primary treatment option.

All patents, patent applications, provisional applications, and publications referred to or cited herein are incorporated by reference in their entirety, including all figures and tables, to the extent they are not inconsistent with the explicit teachings of this specification.

It should be understood that the examples and embodiments described herein are for illustrative purposes only and that various modifications or changes in light thereof will be suggested to persons skilled in the art and are to be included within the spirit and purview of this application and the scope of the appended claims. These examples should not be construed as limiting. In addition, any elements or limitations of any invention or embodiment thereof disclosed herein can be combined with any and/or all other elements or limitations (individually or in any combination) or any other invention or embodiment thereof disclosed herein, and all such combinations are contemplated within the scope of the invention without limitation thereto.

What is claimed:

1. A composition comprising amphotericin B, idebenone or an analog thereof, and, optionally, a pharmaceutically acceptable carrier.

2. The composition of claim 1, consisting of amphotericin B and idebenone and, optionally, a pharmaceutically acceptable carrier.

3. A method of treating an infection caused by a fungal pathogen in a subject, the method comprising administering to the subject an effective amount of amphotericin B and idebenone.

4. The method of claim 3, wherein the fungal pathogen is selected from Candida spp., Malassezia spp., Cryptococcus spp., Aspergillus spp., Nocardia spp., Saccharomyces spp., Microsporum spp, and Trichophyton spp.

5. The method of claim 4, wherein the fungal pathogen is Candida spp.

6. The method of claim 5, wherein the Candida spp. is selected from Candida albicans, Candida auris, Candida glabrata, Candida parapsilosis, Candida dubliensis, Candida tropicalis, Candida lusitaniaem, Candida pseudoguillerimondi, and Candida krusei.

7. The method of claim 5, wherein the fungal pathogen is Candida albicans or Candida auris.

8. The method of claim 4, wherein the fungal pathogen is Cryptococcus spp.

9. The method of claim 8, wherein the fungal pathogen is Cryptococcus neoformans.

10. The method of claim 4, wherein the fungal pathogen is Aspergillus spp.

11. The method of claim 10, wherein the fungal pathogen is Aspergillus fumigatus.

12. The method of claim 3, wherein the fungal pathogen is a multidrug-resistant fungal pathogen.

13. The method of claim 12, wherein the fungal pathogen is resistant to AmB.

14. The method of claim 3, wherein the subject is an immunocompromised individual.

15. A method for restoring or enhancing the fungicidal activity of AmB against fungal pathogens in a subject, the method comprising co-administering idebenone or an analog thereof to the subject in need thereof with AmB.

16. The method of claim 15, wherein the fungal pathogen is selected from Candida spp., Malassezia spp., Cryptococcus spp., Aspergillus spp., Nocardia spp., Saccharomyces spp., Microsporum spp, and Trichophyton spp.

17. The method of claim 15, wherein the fungal pathogen is Candida spp.

18. The method of claim 17, wherein the Candida spp. is selected from Candida albicans, Candida auris, Candida glabrata, Candida parapsilosis, Candida dubliensis, Candida tropicalis, Candida lusitaniaem, Candida pseudoguillerimondi, and Candida krusei.

19. The method of claim 15, wherein the fungal pathogen is Candida albicans, Candida auris, Cryptococcus neoformans, or Aspergillus fumigatus.

20. The method of claim 15, wherein the fungal pathogen is resistant to AmB.
